(19) United States
(12) Patent Application Publication
(10) Pub. No.: US 2024/0221325 A1
(43) Pub. Date: Jul. 4, 2024

(54) SYSTEM AND METHOD FOR LOCATION DETERMINATION USING A VIRTUAL ALIGNMENT TARGET

(71) Applicants: Russell Todd Nevins, Las Vegas, NV (US); David Jon Backstein, Toronto (CA); Bradley H. Nathan, Toronto (CA)

(72) Inventors: Russell Todd Nevins, Las Vegas, NV (US); David Jon Backstein, Toronto (CA); Bradley H. Nathan, Toronto (CA)

(21) Appl. No.: 18/500,997

(22) Filed: Nov. 2, 2023

Related U.S. Application Data
(60) Provisional application No. 63/382,096, filed on Nov. 2, 2022.

(51) Int. Cl. G06T 19/00; A61B 34/20; A61B 90/00; G06T 19/20

(52) U.S. Cl. CPC G06T 19/006; A61B 34/20; A61B 90/37; A61B 90/39; G06T 19/20; A61B 2090/365; A61B 2090/3937; G06T 2219/2004; G06T 2219/2012

(57) ABSTRACT

A system and method for determining a location for a surgical procedure, includes a surgical tool, a 3D spatial mapping device configured to generate map data representative of a three dimensional surface of a bone, and further configured to identify location and orientation data of the surgical tool. The system and method may also include a computer system that receives the map data representative of the three dimensional surface of a bone and the location and orientation data of the surgical tool from the 3D spatial mapping device, and is configured to determine a target location of the surgical tool on the bon. The system and method may also include a mixed reality display, wherein the computer system is configured to send the map data representative of the three dimensional surface of a bone and the location and orientation data of the surgical tool to the mixed reality display, and the mixed reality display is configured to generate a 3D virtual object and a virtual image, wherein the location of the virtual image with respect to the 3D virtual object is correlated to the location of the surgical tool with respect to the target location.

### CROSS-REFERENCE TO RELATED APPLICATIONS

This application claims the benefit of U.S. Provisional Application No. 63/382,096, filed Nov. 2, 2022, which is hereby incorporated by reference herein in its entirety, including but not limited to those portions that specifically appear hereinafter, the incorporation by reference being made with the following exception: In the event that any portion of the above-referenced application is inconsistent with this application, this application supercedes said above-referenced application.

### STATEMENT REGARDING FEDERALLY SPONSORED RESEARCH OR DEVELOPMENT

Not applicable.

### BACKGROUND

### 1. The Field of the Present Disclosure

The present disclosure relates generally to surgical systems and methods of facilitating the efficiency and accuracy of implanting surgical prostheses using a mixed reality display to position a surgical tool in alignment with a target location.

### 2. Description of Related Art

The present disclosure relates generally to surgical systems and methods of facilitating the efficiency and accuracy of implanting surgical prostheses using mixed reality and 3D spatial mapping devices.

### 2. Description of Related Art

In traditional implant surgeries, e.g., knee replacements, a surgeon will utilize a metal jig that is used as a drilling or cutting guide to make the necessary corresponding cuts and holes in the bone of the knee to facilitate placement and attachment of the implant to the bone. However, these metal jigs must be stocked in a variety of different sizes to accommodate different needs and sizes of patients. Accordingly, significant stocks of metal jigs must be stored and sterilized. Additionally, use of such metal jigs in surgical applications is not an exact science and requires the surgeon to manually attach the metal jig to a corresponding bone for use as a drill or cutting guide, which necessarily introduces some inaccuracies.

In knee replacement surgery, the femoral and tibial implants are designed to be surgically implanted into the distal end of the femur and the proximal end of the tibia, respectively. The femoral implant is further designed to cooperate with the tibial implant in simulating the articulating motion of an anatomical knee joint.

These femoral and tibial implants, in combination with associated ligaments and muscles, attempt to duplicate natural knee motion as well as absorb and control forces generated during the range of flexion. In some instances, such as when the femoral or tibial implant becomes excessively worn at the joint, it may be necessary to replace or modify an existing the femoral and/or tibial implant. Such replacements are generally referred to as revision implants.

To prepare a femur and tibia for such a knee replacement operation and to form an engagement with femoral and tibial implants, the femur and tibia bones must be cut in very specific and precise ways and at very specific and precise angles and locations so that the prepared bones will properly engage with and can be properly secured to the corresponding implants. In order to properly cut the femur and tibia, a surgeon traditionally uses a jig or surgical cutting guide as is known to those skilled in the field. For each bone to be cut, a jig is temporarily attached or secured to the bone, such that slots or guides in the jig facilitate the alignment of cutting tools used to make precise cuts necessary for securing a corresponding implant.

The phrase “jig” as used herein, refers broadly to a surgical cutting guide that may be configured and arranged to be fixed or attached to a bone, or secured adjacent to a bone or other tissue, to be cut by a surgeon and identify a relative location, angle and/or cutting plane that a surgeon should cut or drill on the adjacent bone or tissue, as known in the art. A jig may include predetermined slots, apertures, holes and/or cutting surfaces to identify where a surgeon should cut or drill the adjacent bone or tissue, wherein such cuts or holes may correspond to a shape of a surgical implant that may be attached to the cut bone or tissue. A “cutting surface” may refer to a guide edge for guiding the path of a cutting instrument.

Conventional jigs are typically made of a metal alloy and, due to the precise tolerances at which these jigs must be machined, are quite expensive, as high ranging as $40,000-$50,000 in some cases. These metal jigs must also be stored and reused, which adds additional cost and utilizes space resources. Additionally, jigs of various sizes must be kept on hand to accommodate patients of different sizes and with different needs.

In other conventional embodiments, holographic jigs, also referred to as virtual jigs, have been used to enable a surgeon to visualize the positioning and proper sizing of a jig to a bone. However, in use, when the surgeon attempts to superimpose a physical jig over the virtual jig to attach it to a bone to make the required bone cuts, the physical jig will impair the view of the virtual or holographic jig, making it difficult to utilize the holographic jig to accurately place the physical jig.

Accordingly, there is a need for a system and method of utilizing a virtual image to locate and align with a target location using a mixed reality display that can facilitate increased accuracy and precision of required or desired surgical procedures.

The features and advantages of the present disclosure will be set forth in the description which follows, and in part will be apparent from the description, or may be learned by the practice of the present disclosure without undue experimentation. The features and advantages of the present disclosure may be realized and obtained by means of the instruments and combinations particularly pointed out in the appended claims. Any discussion of documents, acts, materials, devices, articles or the like which has been included in the present specification is not to be taken as an admission that any or all of these matters form part of the prior art base, or were common general knowledge in the field relevant to the present disclosure as it existed before the priority date of each claim of this application.

### BRIEF DESCRIPTION OF THE DRAWINGS

The features and advantages of the disclosure will become apparent from a consideration of the subsequent detailed description presented in connection with the accompanying drawings in which:

FIG. 1 is a schematic rendering of a mixed reality system of the present disclosure;

FIG. 2 is a schematic rendering of a view through a mixed reality display of a further embodiment of the present disclosure;

FIG. 3 is another schematic rendering of a view through a mixed reality display of a further embodiment of the present disclosure;

FIG. 4 is a further schematic rendering of a view through a mixed reality display of a further embodiment of the present disclosure;

FIG. 5 is alternative schematic rendering of a view through a mixed reality display of a further embodiment of the present disclosure;

FIG. 6 is a flow diagram illustrating a method of using the mixed reality system; and

FIG. 7 is a flow diagram illustrating another method of using the mixed reality system.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the terms “virtual, ” and “hologram” are used interchangeably, and grammatical equivalents thereof are inclusive or open-ended terms that do not exclude additional, unrecited elements or method steps. These terms are used to describe visual representations of an actual physical device or element, having all, or mostly all, of the same visual characteristics of the physical device, including size and shape.

Applicant has discovered a novel system and method for generating and using a virtual axis, or virtual instrument, in a surgical procedure, for example, in a knee or tibial implant procedure, or other desired surgical procedure.

The phrase “virtual system” as used herein, shall refer broadly to any system capable of generating or creating a simulated or virtual rendering or projection of physical or structural features identical or substantially identical to an actual physical or device, instrument other physical structure, as known in the art. A virtual system may also include a device, mechanism, or instrument capable of projecting or displaying the desired a simulated or virtual rendering or projection of physical or structural features identical or substantially identical to an actual physical device. A virtual system may also enable a user to manipulate, move and/or modify the simulated or virtual rendering or projection.

The phrase “mixed or augmented reality system” as used herein, shall refer broadly to any system capable of generating or creating a simulated or virtual rendering or projection of physical or structural features identical or substantially identical to an actual physical device, instrument or other physical structure, as known in the art. A mixed or augmented reality system may also include a device, mechanism, or instrument capable of projecting or displaying the desired a simulated or virtual rendering or projection of physical or structural features identical or substantially identical to an actual physical device overlaid or concurrently with actual physical structures, mechanism or devices in reality, thus incorporating the virtual rendering or projection in real world settings with actual physical element. A mixed or augmented reality system may also enable a user to manipulate, move and/or modify the simulated or virtual rendering or projection.

The phrase “mixed or augmented reality instrument” as used herein, shall refer broadly to any device, mechanism or instrument used in a mixed or augmented reality system, including a device capable of generating or creating a simulated or virtual rendering or projection of physical or structural features identical or substantially identical to an actual physical device, instrument or other physical structure, as known in the art. A mixed or augmented reality instrument may also be capable of projecting or displaying the desired a simulated or virtual rendering or projection of physical or structural features identical or substantially identical to an actual physical device overlaid or concurrently with actual physical structures, mechanism or devices in reality, thus incorporating the virtual rendering or projection in real world settings with actual physical element. A mixed or augmented reality instrument may also enable a user to manipulate, move and/or modify the simulated or virtual rendering or projection.

The phrase “holographic representation” or “virtual representation” as used herein, shall refer broadly to a visual rendering or projection representing an actual physical device or element, having all, or mostly all, of the same visual characteristics of the corresponding physical device or element, including size and shape, as known in the art.

The phrase “virtual object” or “virtual image” as used herein, shall refer broadly to a visual rendering or projection representing any desired shape or size in two or three dimensions.

The phrase “surgical tool” as used herein, shall refer broadly to a jig, a surgical implant, an operating or medical device, or any other object that may be used during the course of a medical procedure.

The phrase “data representative of a three dimensional surface of a bone” as used herein, when used in the context of generating, capturing, receiving or sending “data representative of a three dimensional surface of a bone, ” shall refer broadly to the concept of generating, capturing, receiving or sending any digital or analog information used to describe or identify the contours, shapes, sizes, and relative locations of surfaces of a bone or other desired object. This data may be relative to multi-dimensional space and/or coordinate systems, including, but not limited to two dimensional (2D) and three dimensional (3D) space and/or coordinate systems. This “data” can also be utilized in, but is not limited to, generating 2D images or scans, which represent a 3D rendering or representation of the bone or other desired object.

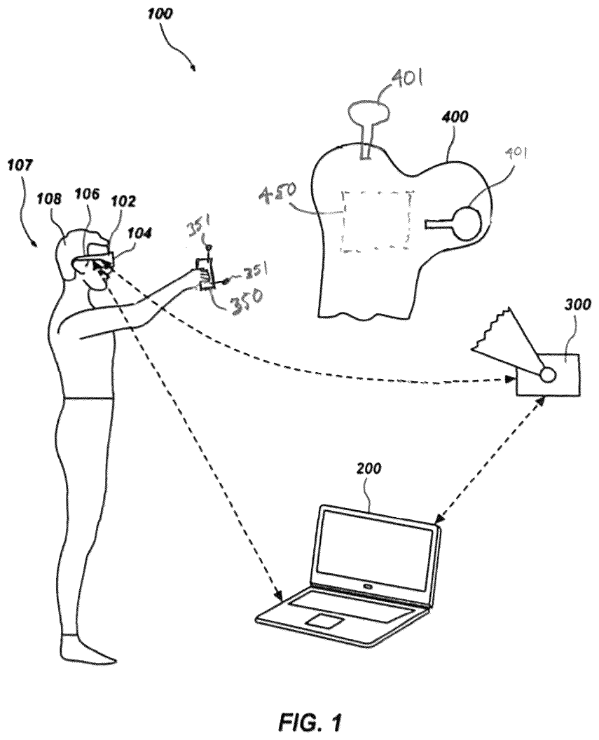

Referring now to FIG. 1, a disclosed embodiment illustrates a mixed or augmented system 100, which can be used to produce, or display, a desired mixed or augmented reality instrument, such as a 3D virtual object and/or a virtual image in a display to a surgeon or user, or stated another way, a virtual object and/or virtual image that is visible and manipulatable by a surgeon or user. The mixed or augmented reality system 100 may also enable a user to activate or deactivate, in full or in part, the 3D virtual objects and/or a virtual images, making the 3D virtual objects and/or a virtual images appear or disappear, as, for example, may be desired in a mixed reality assisted surgery.

The mixed or augmented reality system 100 may include a mixed or augmented reality headset or display 102 which may include a transparent or mostly transparent viewer 104 which can be suspended or positioned in front of a user's eyes. The headset 102 may include a headband 106 attached to the viewer 104, which may be used to secure the headset 102 to a user's head 108, thereby securing the viewer 104 in place in front of the user's eyes.

The transparent viewer 104 may be configured to project, or otherwise make viewable, on an interior surface of the viewer 104, a holographic or virtual image or images, such as a virtual object, for example, a virtual bounding box, a virtual box, a 3D virtual object of any desired shape or size, a virtual image or any desired shape or size, or virtual surgical tool, or a virtual surgical implant, which may be positionally manipulated by the user, surgeon, third party or remote system, such as a remote computer system.

The headset 102 may be configured to view holographic images or, alternatively, the holographic images may be turned off and the user wearing the headset 102 may be able to view the surrounding environment through the transparent viewer 104 without obstruction. As such, a user, such as a surgeon for example, can wear the mixed or augmented reality headset or display 102 and then can choose to activate a holographic image to aide in facilitating a surgical procedure and then shut off the holographic image in order to perform the surgical procedure in a visually un-obscured manner.

One embodiment of the disclosed headset 102 utilizes a Microsoft Corporation product, known as the HoloLens® mixed or augmented reality system. Other suitable mixed or augmented reality systems for generating virtual images viewable by a user or surgeon may also be employed. Thus, the headset 102 may be a conventional “off the shelf” product with a built-in platform that enables all of the features described herein with respect to the headset 102. Furthermore, the headset 102, such as Microsoft's HoloLens® product, can be loaded or preloaded with all desired or required virtual instruments, virtual objects, virtual images, virtual bounding boxes, virtual surgical instruments, virtual surgical implants, virtual jigs or surgical cutting guides, virtual drill bits, and/or a virtual target which can identify relative locations of a surgical procedure, such as where to place a jig on a bone, or where to drill or cut a bone for a surgical procedure, and any other desired virtual instruments or holograms. The Microsoft HoloLens® product and its capabilities and features, or any suitable mixed or augmented reality system, such as is described herein with respect to the headset 102, are known to those skilled in the art.

The mixed reality system 100 may also include a computer or computer system 200 having enabling software to communicate with the headset 102, by both receiving information from the headset 102 and transmitting data and images to the headset 102. It is therefore to be understood, by way of the circuit diagram and dashed lines shown in FIG. 1, that headset 102 is electronically connected to the computer system 200 and an imaging device 300, such as a 3D spatial mapping camera, infrared camera, stereotactic camera, CAT scan device, MRI device or other desired imaging device. In alternative embodiments, the included figures and drawings are schematic in nature and do not disclose the mechanical or electrical details of the imaging devices, as those details are known by those skilled in the art. The imaging device 300 may be electronically connected to the headset 102 and the computer system 200, as shown in the circuit diagram and dashed lines shown in FIG. 1.

One embodiment of the disclosed imaging device may be a 3D spatial mapping camera that may be a product created and manufactured by Microsoft Corporation, known as the Azure Kinect®, or any suitable 3D spatial mapping camera capable of continuous 3D mapping and transition corresponding 3D images, such as bones, anatomy, or other desired 3D objects. The spatial mapping camera may be a conventional “off the shelf” product with a built-in platform that enables all of the features described herein with respect to the spatial mapping camera. Furthermore, the spatial mapping camera, such as Microsoft's Azure Kinect® product, can be loaded or preloaded with all necessary software to enable wireless communication between the imaging device 300 and the computer system 200 and/or the headset 102. Microsoft's Azure Kinect® product and its capabilities and features, or any suitable 3D spatial mapping camera such as is described herein with respect to the imaging device 300, are known to those skilled in the art.

The 3D spatial mapping camera may include sensor software development kits for low-level sensor and device access, body tracking software development kits for tracking bodies or objects in 3D, and speech cognitive services software development kits for enabling microphone access and cloud-based or wireless speech services.

Additionally, the spatial mapping camera 300 may include the following features: depth camera access and mode control (a passive IR mode, plus wide and narrow field-of-view depth modes); RGB camera access and control (for example, exposure and white balance); a motion sensor (gyroscope and accelerometer) access; synchronized depth-RGB camera streaming with configurable delay between cameras; external device synchronization control with configurable delay offset between devices; camera frame meta-data access for image resolution, timestamp, etc.; and device calibration data access.

The spatial mapping camera may also include: software development kits that enable a viewer tool to monitor device data streams and configure different modes; a sensor recording tool and playback reader API that uses the Matroska container format, as known to those of ordinary skill in the field; a library and runtime to track bodies in 3D when used with the spatial mapping camera 300; an anatomically correct skeleton for each partial or full body; a unique identity for each body; ability to track bodies over time; and speech recognition and translation capabilities, such as, speech-to-text, speech translation and text-to-speech.

In one disclosed embodiment, the imaging device 300 comprises an infrared camera, stereotactic camera, CAT scan device, or MRI device, or any imagining device which may be suitable and capable of capable of continuous imaging, such as bones, anatomy, or other desired 3D objects. These imaging devices may be a conventional “off the shelf” products with a built-in platform that enables all of the features described herein. Furthermore, the imaging device can be loaded or preloaded with all necessary software to enable wireless communication between the imaging devices 300 and the computer system 200 and/or the headset 102. These imaging devices, as known in the art, may provide or identify desired coordinates, orientation, and/or mapping data in a 3D space, thus providing accurate and detailed location determination in a desired 3D space.

The headset 102, computer system 200, imaging device 300, may be programmed and configured to enable a surgeon 107 to see and manipulate a virtual image, virtual object, or holographic target or jig, or visual representation of a jig, corresponding to a physical surgical tool and a target location, such as a desired location for a surgical procedure on a patient's bone or tissue, or any other desired location, which may receive a surgical procedure. The headset 102, computer system 200 and imaging device 300 may communicate with one another via a local network connection, Wi-Fi, Bluetooth®, or any other known wireless communication signal.

Specifically, the imaging device 300, which may be programed to communicate with the computer system 200 having enabling software, may utilize such enabling software to the location and movements of a surgical tool 350 with respect to a surgical location, such a patient's bone 400. The imaging device 300 may collect three dimensional mapping, location and orientation data from the bone 400 and the enabling software may identify a target location for the desired surgical procedure, prior to cutting or drilling the bone 400.

As shown in FIGS. 1-5, the computer system 200 with enabling software may utilize the data and respective images, coordinates, and/or imaging information or display data from the imaging camera 300, to pair and/or correlate the movements and location of a virtual image 500 with the movements and location of the surgical tool 350.

The computer system 200 with enabling software may also utilize the data and respective images, coordinates, and/or imaging information or display data from the imaging camera 300, to pair and/or correlate the location and orientation of a target location 450 of the surgical procedure on the bone 400 with a 3D virtual object 600.

The computer system 200 with enabling software can then send data and information related to the virtual image 500 and the 3D virtual object to the mixed reality headset 102, sch that the user 107 can see the virtual image 500 and the 3D virtual object in the mixed reality headset 102.

As the user 107 moves the surgical tool 350 with respect to the target location 450 on the bone 400, the virtual image 500 will correspondingly move with respect to the 3D virtual object. The computer system 200 with enabling software can pair and/or correlate the spatial relationship between the virtual image 500 and the virtual object 600 with the surgical tool 350 and the target location 450, such that when the virtual image 500 is moved or manipulated inside of the virtual object 600, such that the substantial entirety of the virtual image 500 lies within the virtual object 600, then the surgical tool 350 is precisely aligned with the target location 450 on the bone 400. Said another way, as the surgical tool 350 is moved into proper alignment with the target location 450, the virtual image 500 will move within the virtual object 600. When the surgical tool is in the precise, or substantially precise, alignment with the target location 450, the virtual image 500 will be entirely within the virtual object 600.

The surgical tool 350 may be or include a surgical implant, jig, or operating tool, or any other desired tool or instrument that needs to be fixed, used or otherwise utilized at a specific location, such as a target location 450. The surgical tool 350 may include a marker 351, or markers, which may be attached to the surgical tool 350, making it easer for the imaging device 300 to identify and track the exact location of the surgical tool in space and with respect to the target location 450. The marker 351 may be a fiducial ball, QR label, or other small identifier. Due to the high accuracy and resolution of the imaging device 300, a small fiducial-type marker or QR label may be all that is necessary to clearly and accurately identify the location of the surgical tool 350.

Similarly, the bone 400, or surrounding tissue, may also include a marker 451, or markers, which may be attached to the bone 400, or surrounding tissue, making it easer for the imaging device 300 to identify and track the exact location of the bone 400 in space. The software enabled computer system can be preloaded, or inputted with, the precise location of the target location 450 on the bone 400 utilizing the marker (s) 451. The marker 351 may be a fiducial ball, QR label, or other small identifier. Due to the high accuracy and resolution of the imaging device 300, a small fiducial-type marker or QR label may be all that is necessary to clearly and accurately identify the location of the bone 400, and thereby the target location 450, in space and with respect to the surgical tool 350.

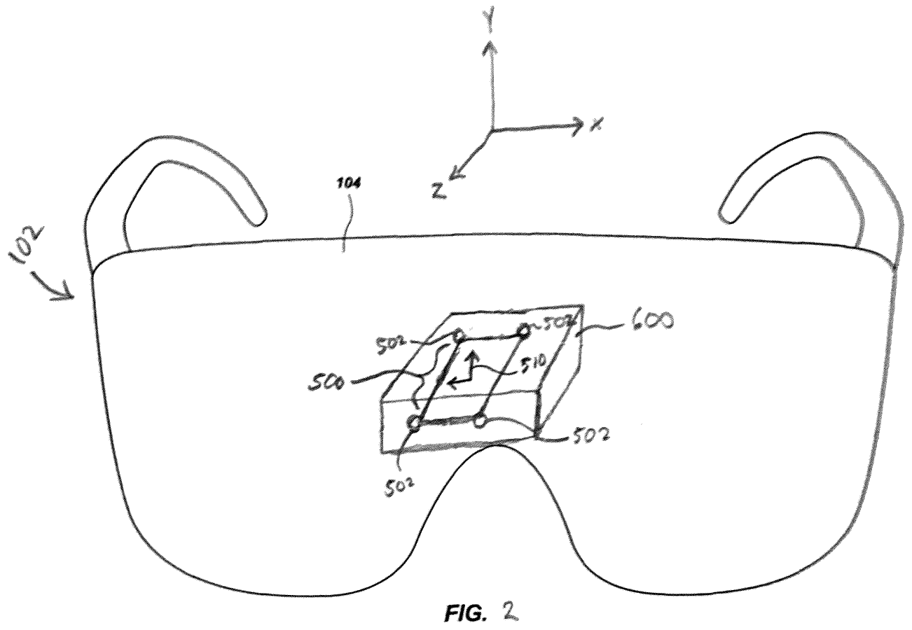

As illustrated in FIGS. 2-5, the virtual image 500, may be rectangular in shape, or be formed in any other desired shape. The virtual image 500 may also include one or more identifiers 502 located at fixed, predetermined locations on the virtual image 500. For example, the identifies my be fixed to corners of the virtual image 500. As the virtual image 500 is manipulated and move inside of the virtual object 600 the corresponding identifier 502 can change colors, for example, red to green, when the portion of the virtual image closest to the corresponding identifier is within the virtual object 600. This change in color of the corresponding identifiers can provide an additional visual indicator to the user 107 that the virtual image 500 is within or outside of the virtual object 600. Once all of the identifiers have changed colors, the user can know that the virtual image is fully within the virtual object 600, and thereby the corresponding surgical tool 350 is aligned with the target location 450.

A vertical and horizontal axis indicator 510 may also be fixed to a location on the virtual image, such that the corresponding horizontal or vertical axis 510 may change colors, for example, red to green, when the corresponding horizontal and/or vertical angle of the virtual image 500 is within the proper and desired angular alignment with the virtual object 600, and thereby the corresponding surgical tool 350 is aligned with the target location 450.

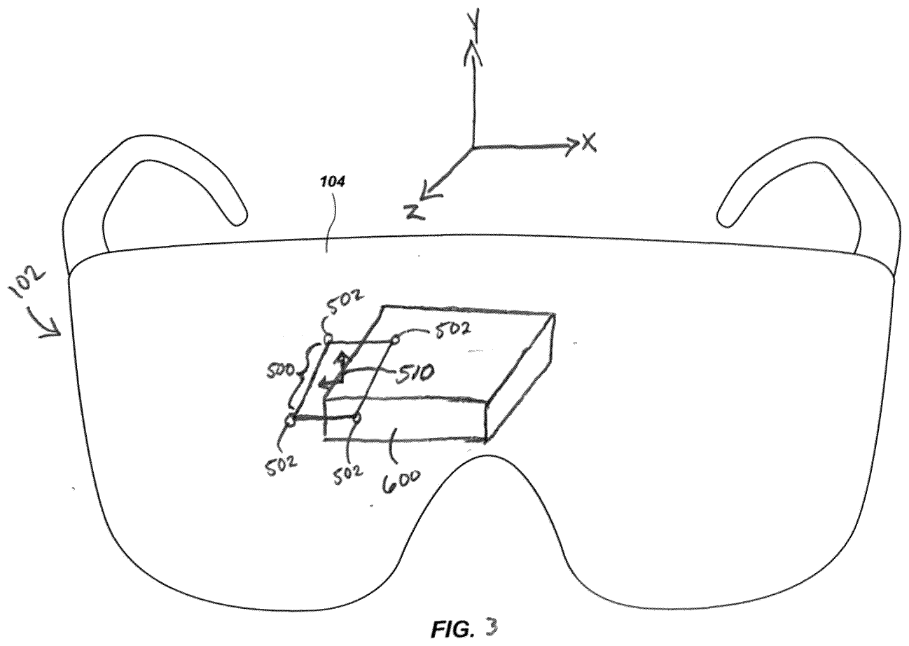

FIG. 2 illustrates the virtual image 500 fully within the virtual object 600, and thus the corresponding surgical tool 350 (not shown in FIG. 3) would be in precise alignment with the target location 450.

FIG. 3 illustrates the virtual image 500 is not fully within the virtual object 600 and thus the corresponding surgical tool 350 (not shown in FIG. 3) would not be in precise alignment with the target location 450.

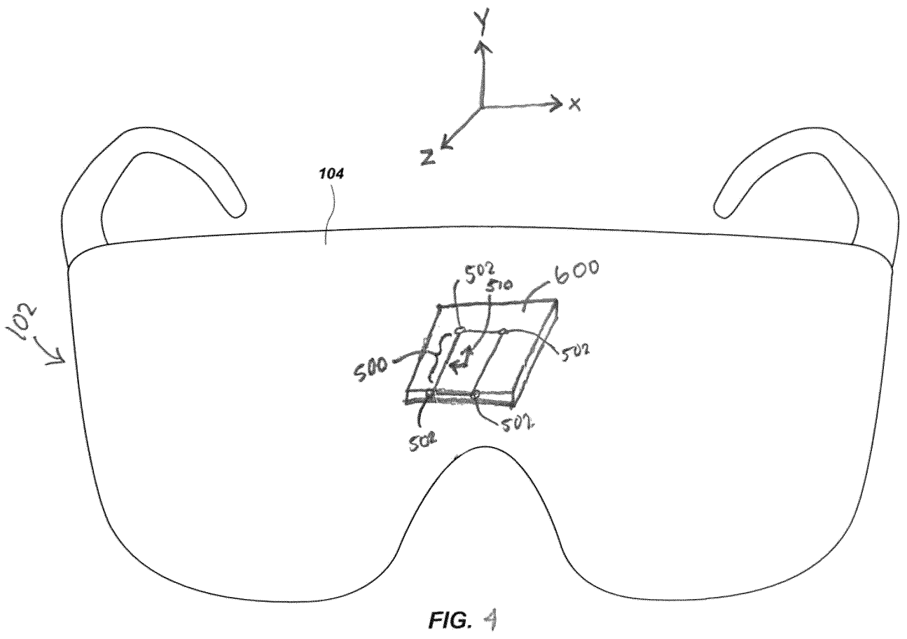

In another embodiment, as shown in FIG. 4, a user can voluntarily reduce or change the relative size of the virtual object 600 to further refine the alignment of the virtual image 500 within the virtual object 600. This may be advantageous in situations where a greater location tolerance could be used initially to move the virtual image 500 into the virtual object 600, and then the tolerance, and thereby the relative size of the virtual object 600, could be manipulated and changed to further reduce the relative tolerance between the virtual image 500 and the virtual object 600. Refining the tolerances in this way may also increase the relative tolerance between the surgical tool 350 and the target location 450.

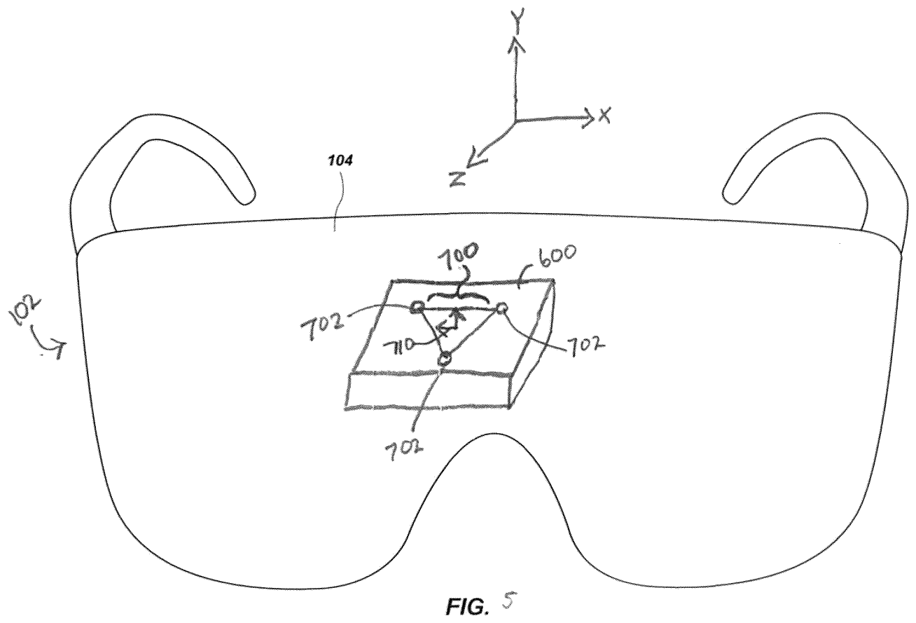

In another embodiment, as shown in FIG. 5. A virtual image 700 may have a different shape, such as triangular for example. The virtual image 700 can still be used in the same methodology as discussed above with respect to virtual image 500, and the virtual object 600 does not need to mirror the same relative shape as the virtual image 700, as long as the virtual image can still fully lie within the virtual object. In this embodiment, the virtual image 700 can still include corresponding indicators 702 and axis indicator 710, which would function in the same way as indicators 502 and axis indicator 510, as discussed above.

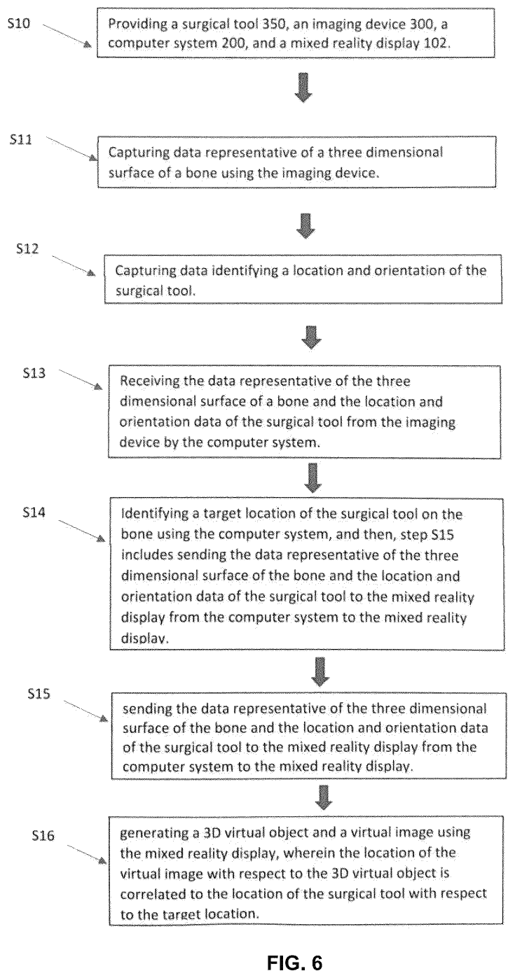

FIG. 6 illustrates a flow diagram, used to show the method of using the mixed reality system 100 according to an exemplary embodiment. The method of using the system 100 in a first step S10, provides a surgical tool 350, an imaging device 300, a computer system 200, and a mixed reality display 102.

The next step S11 includes capturing data representative of a three dimensional surface of a bone using the imaging device.

Then step S12 includes capturing data identifying a location and orientation of the surgical tool.

Step S13 includes receiving the data representative of the three dimensional surface of a bone and the location and orientation data of the surgical tool from the imaging device by the computer system.

Then step S14 includes identifying a target location of the surgical tool on the bone using the computer system, and then, step S15 includes sending the data representative of the three dimensional surface of the bone and the location and orientation data of the surgical tool to the mixed reality display from the computer system to the mixed reality display.

Step S16 then includes generating a 3D virtual object and a virtual image using the mixed reality display, wherein the location of the virtual image with respect to the 3D virtual object is correlated to the location of the surgical tool with respect to the target location.

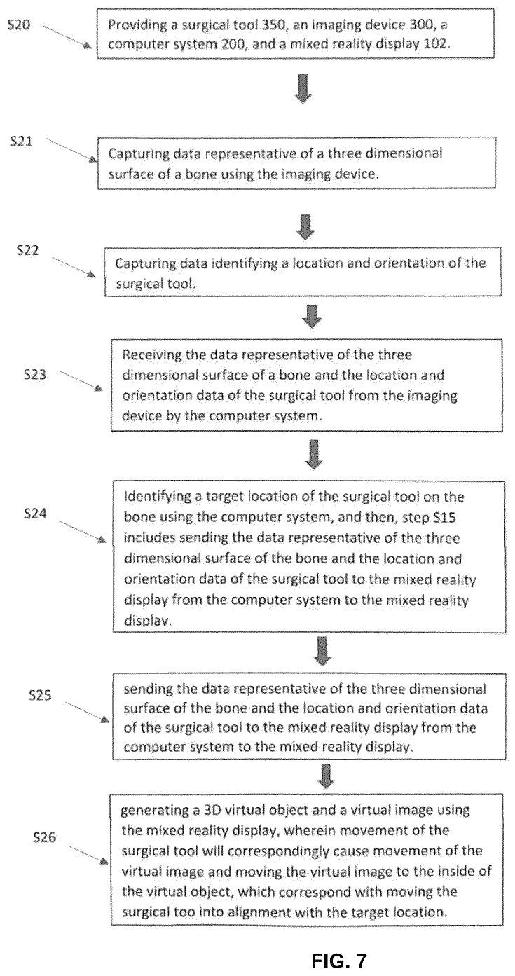

FIG. 7 illustrates a flow diagram of another embodiment, used to show a method of using the mixed reality system 100. The method of using the system 100 in a first step S20, provides a surgical tool 350, an imaging device 300, a computer system 200, and a mixed reality display 102.

The next step S21 includes capturing data representative of a three dimensional surface of a bone using the imaging device.

Then step S22 includes capturing data identifying a location and orientation of the surgical tool.

Step S23 includes receiving the data representative of the three dimensional surface of a bone and the location and orientation data of the surgical tool from the imaging device by the computer system.

Then step S24 includes identifying a target location of the surgical tool on the bone using the computer system, and then, step S25 includes sending the data representative of the three dimensional surface of the bone and the location and orientation data of the surgical tool to the mixed reality display from the computer system to the mixed reality display.

Step S26 then includes generating a 3D virtual object and a virtual image using the mixed reality display, wherein movement of the surgical tool will correspondingly cause movement of the virtual image and moving the virtual image to the inside of the virtual object, which correspond with moving the surgical too into alignment with the target location.

It is to be understood that the various embodiments disclosed and described above and shown in the accompanying figures, may be interchangeably sued together, independently or in any desired combination of disclosed features.

### Additional Specification Support

Embodiment 1. A system for determining a location for a surgical procedure, including: a surgical tool; a 3D spatial mapping device, wherein the 3D spatial mapping device is configured to generate map data representative of a three dimensional surface of a bone, and further configured to identify location and orientation data of the surgical tool; a computer system that receives the map data representative of the three dimensional surface of a bone and the location and orientation data of the surgical tool from the 3D spatial mapping device, and wherein the computer system is configured to determine a target location of the surgical tool on the bone; and a mixed reality display, wherein the computer system is configured to send the map data representative of the three dimensional surface of a bone and the location and orientation data of the surgical tool to the mixed reality display, and the mixed reality display is configured to generate a 3D virtual object and a virtual image, wherein the location of the virtual image with respect to the 3D virtual object is correlated to the location of the surgical tool with respect to the target location.

Embodiment 2. The system of embodiment 1, wherein a user can manipulate the location of the virtual image with respect to the 3D virtual object by moving the surgical tool.

Embodiment 3. The system of embodiment 1, wherein the surgical tool is a jig.

Embodiment 4. The system of embodiment 1, wherein the surgical tool is an implant.

Embodiment 5. The system of embodiment 1, further comprising,
at least one marker attached to the bone.

Embodiment 6. The system of embodiment 5, wherein the marker attached to the bone is a fiducial ball.

Embodiment 7. The system of embodiment 1, further including, at least one marker attached to the surgical tool.

Embodiment 8. The system of embodiment 7, wherein the marker attached to the surgical tool is a fiducial ball.

Embodiment 9. The system of embodiment 1, wherein a user can manipulate the location of the virtual image by moving the surgical tool with respect to the target location.

Embodiment 10. The system of embodiment 1, wherein the correlation between the location of the virtual image with respect to the 3D virtual object and the location of the surgical tool with respect to the target location, further includes when the surgical tool has reached the target location, the virtual image will be located within the 3D virtual object.

Embodiment 11. The system of embodiment 1, wherein at least a portion of the virtual image will change color when the portion of the virtual image is within the 3D virtual object.

Embodiment 12. The system of embodiment 1, wherein a size of the 3D virtual object may be manipulated by the user to refine the accuracy of the correlation between the location of the virtual image with respect to the 3D virtual object and the location of the surgical tool with respect to the target location.

Embodiment 11. The system of embodiment 1, wherein the 3D virtual object is formed in the shape of a box.

Embodiment 12. The system of embodiment 1, wherein the virtual image may include a virtual axis that will change color when the virtual image reaches a target angular position.

Embodiment 13. A system for determining a location for a surgical procedure, including: a surgical tool; an imaging device, wherein the imaging device is configured to generate data representative of a three dimensional surface of a bone, and further configured to identify location and orientation data of the surgical tool; a computer system that receives the data representative of the three dimensional surface of a bone and the location and orientation data of the surgical tool from the imaging device, and wherein the computer system is configured to determine a target location of the surgical tool on the bone; and a mixed reality display, wherein the computer system is configured to send the data representative of the three dimensional surface of a bone and the location and orientation data of the surgical tool to the mixed reality display, and the mixed reality display is configured to generate a 3D virtual object and a virtual image, wherein the location of the virtual image with respect to the 3D virtual object is correlated to the location of the surgical tool with respect to the target location, and wherein a user can manipulate the location of the virtual image with respect to the 3D virtual object by moving the surgical tool.

Embodiment 14. A method for determining a location for a surgical procedure, including: providing a surgical tool; providing an imaging device; providing a computer system; providing a mixed display; reality capturing data representative of a three dimensional surface of a bone using the imaging device; capturing data identifying a location and orientation of the surgical tool; receiving the data representative of the three dimensional surface of a bone and the location and orientation data of the surgical tool from the imaging device by the computer system; identifying a target location of the surgical tool on the bone using the computer system; sending the data representative of the three dimensional surface of the bone and the location and orientation data of the surgical tool to the mixed reality display from the computer system to the mixed reality display; and generating a 3D virtual object and a virtual image using the mixed reality display, wherein the location of the virtual image with respect to the 3D virtual object is correlated to the location of the surgical tool with respect to the target location.

Embodiment 15. The method of embodiment 14, wherein a user can manipulate the location of the virtual image with respect to the 3D virtual object.

Embodiment 16. The method of embodiment 14, wherein a user can manipulate the location of the virtual image by moving the surgical tool with respect to the target location.

Embodiment 17. The method of embodiment 14, wherein the correlation between the location of the virtual image with respect to the 3D virtual object and the location of the surgical tool with respect to the target location, further includes when the surgical tool has reached the target location, the virtual image will be located within the 3D virtual object.

Embodiment 18. The method of embodiment 14, wherein at least a portion of the virtual image will change color when the portion of the virtual image is within the 3D virtual object.

Embodiment 19. The method of embodiment 14, wherein a size of the 3D virtual object may be manipulated by the user to refine the accuracy of the correlation between the location of the virtual image with respect to the 3D virtual object and the location of the surgical tool with respect to the target location.

Embodiment 20. The method of embodiment 14, wherein the 3D virtual object is formed in the shape of a box.

Embodiment 21. The method of embodiment 14, wherein the virtual image may include a virtual axis that will change color when the virtual image reaches a target angular position.

Embodiment 22. A method for determining a location for a surgical procedure, including: providing a surgical tool; providing an imaging device; providing a computer system; providing a mixed reality display; capturing data representative of a three dimensional surface of a bone using the imaging device, capturing data identifying a location and orientation of the surgical tool; receiving the data representative of the three dimensional surface of a bone and the location and orientation data of the surgical tool from the imaging device by the computer system, identifying a target location of the surgical tool on the bone using the computer system; sending the data representative of the three dimensional surface of the bone and the location and orientation data of the surgical tool to the mixed reality display from the computer system to the mixed reality display, generating a 3D virtual object and a virtual image using the mixed reality display, wherein movement of the surgical tool will correspondingly cause movement of the virtual image; and moving the virtual image to the inside of the virtual object, which correspond with moving the surgical too into alignment with the target location.

Embodiment 23. A system for determining a location for a surgical procedure, including: a surgical tool wherein the surgical tool is a jig; a 3D spatial mapping device, wherein the 3D spatial mapping device is configured to generate map data representative of a three dimensional surface of a bone, and further configured to identify location and orientation data of the surgical tool; a computer system that receives the map data representative of the three dimensional surface of a bone and the location and orientation data of the surgical tool from the 3D spatial mapping device, and wherein the computer system is configured to determine a target location of the surgical tool on the bone; a mixed reality display, wherein the computer system is configured to send the map data representative of the three dimensional surface of a bone and the location and orientation data of the surgical tool to the mixed reality display, and the mixed reality display is configured to generate a 3D virtual object and a virtual image, wherein the location of the virtual image with respect to the 3D virtual object is correlated to the location of the surgical tool with respect to the target location, wherein a user can manipulate the location of the virtual image with respect to the 3D virtual object by moving the surgical tool, at least one marker attached to the bone, wherein the marker attached to the bone is a fiducial ball; at least one marker attached to the surgical tool, wherein the marker attached to the surgical tool is a fiducial ball; wherein a user can manipulate the location of the virtual image by moving the surgical tool with respect to the target location; wherein the correlation between the location of the virtual image with respect to the 3D virtual object and the location of the surgical tool with respect to the target location, further includes when the surgical tool has reached the target location, the virtual image will be located within the 3D virtual object; wherein at least a portion of the virtual image will change color when the portion of the virtual image is within the 3D virtual object; wherein a size of the 3D virtual object may be manipulated by the user to refine the accuracy of the correlation between the location of the virtual image with respect to the 3D virtual object and the location of the surgical tool with respect to the target location; wherein the 3D virtual object is formed in the shape of a box; and wherein the virtual image may include a virtual axis that will change color when the virtual image reaches a target angular position.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

What is claimed:

1. A system for determining a location for a surgical procedure, comprising:
a surgical tool;
a spatial mapping device, wherein the spatial mapping device is configured to generate map data representative of a surface of a bone, and further configured to identify location and orientation data of the surgical tool;
a computer system that receives the map data representative of the surface of a bone and the location and orientation data of the surgical tool from the spatial mapping device, and wherein the computer system is configured to determine a target location of the surgical tool on the bone; and
a mixed reality display, wherein the computer system is configured to send the map data representative of the surface of the bone and the location and orientation data of the surgical tool to the mixed reality display, and the mixed reality display is configured to generate a virtual object and a virtual image, wherein the location of the virtual image with respect to the virtual object is correlated to the location of the surgical tool with respect to the target location.

2. The system of claim 1, wherein the spatial mapping device is a multi-dimensional spatial mapping device, and wherein the data representative of a surface of the bone is data representative of a multi-dimensional surface of the bone, and wherein the virtual object is a multi-dimensional virtual object.

3. The system of claim 2, wherein the multi-dimensional spatial mapping device is a 3D spatial mapping device, and wherein the data representative of a multi-dimensional surface of the bone is data representative of a 3D surface of the bone, and wherein the multi-dimensional virtual object is a 3D virtual object.

4. The system of claim 1, wherein a user can manipulate the location of the virtual image with respect to the 3D virtual object by moving the surgical tool.

5. The system of claim 1, wherein the surgical tool is a jig.

6. The system of claim 1, wherein the surgical tool is an implant.

7. The system of claim 1, further comprising,
at least one marker attached to the bone.

8. The system of claim 7, wherein the marker attached to the bone is a fiducial ball.

9. The system of claim 1, wherein a user can manipulate the location of the virtual image by moving the surgical tool with respect to the target location.

10. The system of claim 3, wherein the correlation between the location of the virtual image with respect to the 3D virtual object and the location of the surgical tool with respect to the target location, further includes when the surgical tool has reached the target location, the virtual image will be located within the 3D virtual object.

11. The system of claim 3, wherein at least a portion of the virtual image will change color when the portion of the virtual image is within the 3D virtual object.

12. The system of claim 3, wherein a size of the 3D virtual object may be manipulated by the user to refine the accuracy of the correlation between the location of the virtual image with respect to the 3D virtual object and the location of the surgical tool with respect to the target location.

13. The system of claim 3, wherein the 3D virtual object is formed in the shape of a box.

14. The system of claim 3, wherein the virtual image may include a virtual axis that will change color when the virtual image reaches a target angular position.

15. A system for determining a location for a surgical procedure, comprising:
a surgical tool;
an imaging device, wherein the imaging device is configured to generate data representative of a three dimensional surface of a bone, and further configured to identify location and orientation data of the surgical tool;
a computer system that receives the data representative of the surface of a bone and the location and orientation data of the surgical tool from the imaging device, and wherein the computer system is configured to determine a target location of the surgical tool on the bone; and
a mixed reality display, wherein the computer system is configured to send the data representative of the surface of the bone and the location and orientation data of the surgical tool to the mixed reality display, and the mixed reality display is configured to generate a virtual object and a virtual image, wherein the location of the virtual image with respect to the virtual object is correlated to the location of the surgical tool with respect to the target location, and wherein a user can manipulate the location of the virtual image with respect to the virtual object by moving the surgical tool.

16. The system of claim 15, wherein the imaging device is a multi-dimensional spatial mapping device, and wherein the data representative of a surface of the bone is data representative of a multi-dimensional surface of the bone, and wherein the virtual object is a multi-dimensional virtual object.

17. The system of claim 16, wherein the multi-dimensional spatial mapping device is a 3D spatial mapping device, and wherein the data representative of a multi-dimensional surface of the bone is data representative of a 3D surface of the bone, and wherein the multi-dimensional virtual object is a 3D virtual object.

18. The system of claim 17, wherein a user can manipulate the location of the virtual image with respect to the 3D virtual object by moving the surgical tool.

19. The system of claim 15, wherein the surgical tool is a jig.

20. The system of claim 15, wherein the surgical tool is an implant.

21. The system of claim 15, further comprising,
at least one marker attached to the bone.

22. The system of claim 21, wherein the marker attached to the bone is a fiducial ball.

23. The system of claim 15, wherein a user can manipulate the location of the virtual image by moving the surgical tool with respect to the target location.

24. The system of claim 17, wherein the correlation between the location of the virtual image with respect to the 3D virtual object and the location of the surgical tool with respect to the target location, further includes when the surgical tool has reached the target location, the virtual image will be located within the 3D virtual object.

25. The system of claim 17, wherein at least a portion of the virtual image will change color when the portion of the virtual image is within the 3D virtual object.

26. The system of claim 17, wherein a size of the 3D virtual object may be manipulated by the user to refine the accuracy of the correlation between the location of the virtual image with respect to the 3D virtual object and the location of the surgical tool with respect to the target location.

27. The system of claim 17, wherein the 3D virtual object is formed in the shape of a box.

28. The system of claim 17, wherein the virtual image may include a virtual axis that will change color when the virtual image reaches a target angular position.

29. A method for determining a location for a surgical procedure, comprising:
providing a surgical tool;
providing an imaging device;
providing a computer system;
providing a mixed reality display;
capturing data representative of a surface of a bone using the imaging device;
capturing data identifying a location and orientation of the surgical tool;
receiving the data representative of the surface of a bone and the location and orientation data of the surgical tool from the imaging device by the computer system;
identifying a target location of the surgical tool on the bone using the computer system;
sending the data representative of the surface of the bone and the location and orientation data of the surgical tool to the mixed reality display from the computer system to the mixed reality display; and
generating a virtual object and a virtual image using the mixed reality display, wherein the location of the virtual image with respect to the virtual object is correlated to the location of the surgical tool with respect to the target location.

30. The method of claim 29, wherein the imaging device is a multi-dimensional spatial mapping device, and wherein the data representative of a surface of the bone is data representative of a multi-dimensional surface of the bone, and wherein the virtual object is a multi-dimensional virtual object.

31. The method of claim 30, wherein the multi-dimensional spatial mapping device is a 3D spatial mapping device, and wherein the data representative of a multi-dimensional surface of the bone is data representative of a 3D surface of the bone, and wherein the multi-dimensional virtual object is a 3D virtual object.

32. The method of claim 31, wherein a user can manipulate the location of the virtual image with respect to the 3D virtual object.

33. The method of claim 29, wherein a user can manipulate the location of the virtual image by moving the surgical tool with respect to the target location.

34. The method of claim 31, wherein the correlation between the location of the virtual image with respect to the 3D virtual object and the location of the surgical tool with respect to the target location, further includes when the surgical tool has reached the target location, the virtual image will be located within the 3D virtual object.

35. The method of claim 31, wherein at least a portion of the virtual image will change color when the portion of the virtual image is within the 3D virtual object.

36. The method of claim 31, wherein a size of the 3D virtual object may be manipulated by the user to refine the accuracy of the correlation between the location of the virtual image with respect to the 3D virtual object and the location of the surgical tool with respect to the target location.

37. The method of claim 31, wherein the 3D virtual object is formed in the shape of a box.

38. The method of claim 29, wherein the virtual image may include a virtual axis that will change color when the virtual image reaches a target angular position.

39. A method for determining a location for a surgical procedure, comprising:
providing a surgical tool;
providing an imaging device;
providing a computer system;
providing a mixed reality display;
capturing data representative of a dimensional surface of a bone using the imaging device,
capturing data identifying a location and orientation of the surgical tool;
receiving the data representative of the dimensional surface of a bone and the location and orientation data of the surgical tool from the imaging device by the computer system,
identifying a target location of the surgical tool on the bone using the computer system;
sending the data representative of the dimensional surface of the bone and the location and orientation data of the surgical tool to the mixed reality display from the computer system to the mixed reality display,
generating a virtual object and a virtual image using the mixed reality display, wherein movement of the surgical tool will correspondingly cause movement of the virtual image; and
moving the virtual image to the inside of the virtual object, which correspond with moving the surgical too into alignment with the target location.

40. The method of claim 39, wherein the imaging device is a multi-dimensional spatial mapping device, and wherein the data representative of a surface of the bone is data representative of a multi-dimensional surface of the bone, and wherein the virtual object is a multi-dimensional virtual object.

41. The method of claim 40, wherein the multi-dimensional spatial mapping device is a 3D spatial mapping device, and wherein the data representative of a multi-dimensional surface of the bone is data representative of a 3D surface of the bone, and wherein the multi-dimensional virtual object is a 3D virtual object.

42. The method of claim 41, the location of the virtual image with respect to the 3D virtual object is correlated to the location of the surgical tool with respect to the target location.

43. The method of claim 41, wherein a user can manipulate the location of the virtual image with respect to the 3D virtual object.

44. The method of claim 39, wherein a user can manipulate the location of the virtual image by moving the surgical tool with respect to the target location.

45. The method of claim 41, wherein the correlation between the location of the virtual image with respect to the 3D virtual object and the location of the surgical tool with respect to the target location, further includes when the surgical tool has reached the target location, the virtual image will be located within the 3D virtual object.

46. The method of claim 41, wherein at least a portion of the virtual image will change color when the portion of the virtual image is within the 3D virtual object.

47. The method of claim 41, wherein a size of the 3D virtual object may be manipulated by the user to refine the accuracy of the correlation between the location of the virtual image with respect to the 3D virtual object and the location of the surgical tool with respect to the target location.

48. The method of claim 41, wherein the 3D virtual object is formed in the shape of a box.

49. The method of claim 39, wherein the virtual image may include a virtual axis that will change color when the virtual image reaches a target angular position.

50. A system for determining a location for a surgical procedure, comprising:
a surgical tool wherein the surgical tool is a jig;
a 3D spatial mapping device, wherein the 3D spatial mapping device is configured to generate map data representative of a three dimensional surface of a bone, and further configured to identify location and orientation data of the surgical tool;
a computer system that receives the map data representative of the three dimensional surface of a bone and the location and orientation data of the surgical tool from the 3D spatial mapping device, and wherein the computer system is configured to determine a target location of the surgical tool on the bone;
a mixed reality display, wherein the computer system is configured to send the map data representative of the three dimensional surface of a bone and the location and orientation data of the surgical tool to the mixed reality display, and the mixed reality display is configured to generate a 3D virtual object and a virtual image, wherein the location of the virtual image with respect to the 3D virtual object is correlated to the location of the surgical tool with respect to the target location, wherein a user can manipulate the location of the virtual image with respect to the 3D virtual object by moving the surgical tool,
at least one marker attached to the bone, wherein the marker attached to the bone is a fiducial ball;
at least one marker attached to the surgical tool, wherein the marker attached to the surgical tool is a fiducial ball;
wherein a user can manipulate the location of the virtual image by moving the surgical tool with respect to the target location;
wherein the correlation between the location of the virtual image with respect to the 3D virtual object and the location of the surgical tool with respect to the target location, further includes when the surgical tool has reached the target location, the virtual image will be located within the 3D virtual object;
wherein at least a portion of the virtual image will change color when the portion of the virtual image is within the 3D virtual object;
wherein a size of the 3D virtual object may be manipulated by the user to refine the accuracy of the correlation between the location of the virtual image with respect to the 3D virtual object and the location of the surgical tool with respect to the target location;
wherein the 3D virtual object is formed in the shape of a box; and
wherein the virtual image may include a virtual axis that will change color when the virtual image reaches a target angular position.
